(19) United States
(12) Patent Application Publication
(10) Pub. No.: US 2005/0020553 A1
(43) Pub. Date: Jan. 27, 2005

(54) Prevention of miscarriage with immunomodulating non-endogenous gestagen compounds

(75) Inventors: Jeannette Alt, Burgwedel (DE); Bernd Eisele, Laatzen (DE)

Correspondence Address: CROWELL & MORING LLP;INTELLECTUAL PROPERTY GROUP, P.O. BOX 14300, WASHINGTON, DC, 20044-4300, US

(73) Assignee: Solvay Pharmaceuticals GmbH, Hannover (DE)

(21) Appl. No.: 10/855,789

(22) Filed: May 28, 2004

Related U.S. Application Data
(63) Continuation of application No. PCT/EP02/13288, filed on Nov. 26, 2002.
(60) Provisional application No. 60/334,124, filed on Nov. 30, 2001.

(51) Int. Cl. A61K 31/56

(52) U.S. Cl. 514/182

(57) ABSTRACT

A method of inhibiting spontaneous or habitual miscarriage by administering to a female patient in need thereof an effective amount of at least one immunologically active non-endogenous gestagen compound.

### CROSS REFERENCE TO RELATED APPLICATIONS

This application is a continuation of international patent application no. PCT EP02/13288, filed Nov. 26, 2002 designating the United States of America and published in English as WO 03/045361, the entire disclosure of which is incorporated herein by reference. Priority is claimed based on U.S. provisional patent application No. 60/334,124, filed Nov. 30, 2001, the disclosure of which is also incorporated herein by reference.

### BACKGROUND OF THE INVENTION

Spontaneous miscarriage (abortion) occurs in 15% of all clinically recognised pregnancies. It is defined by the World Health Organisation (WHO, 1977) as “the expulsion or extraction from its mother of an embryo or fetus weighing 500 g or less”. It is the most common complication in pregnancy. 50-60% of spontaneous pregnancy losses can be explained by chromosomal anomalies of the fetus, infectious etiologies or maternal endocrinologic or anatomic comorbidities. But still 40-50% of spontaneous pregnancy losses remain “unexplained”.

In unexplained cases of pregnancy loss, the most practical and promising way of therapeutic intervention is the administration of dydrogesterone (Duphaston®). Dydrogesterone is an orally active progestogen (=non-endogenous gestagen compound). Over the years, several clinical trials showed the efficacy of dydrogesterone in the treatment of habitual and threatened abortion. Currently Duphaston® is available in over 90 countries and has a cumulative exposure of over 26 million patient years. Approximately 7 million patients have been treated with dydrogesterone with highly probable or proven in-utero exposure.

Until recently, recurrent pregnancy loss in “unexplained cases” was seen as an implantation failure due to a non-receptive out-of-phase endometrium. A luteal phase defect as a condition in which inadequate production of progesterone by the corpus luteum takes place was originally proposed as explanation of otherwise inexplicable cases of infertility. The beneficial influence of dydrogesterone on women that were exposed to an elevated risk of undergoing spontaneous or habitual miscarriage has therefore been regarded as a simple substitution of deficient endogenous progesterone. Consequently, dydrogesterone and other progestogens are currently prescribed only after medical identification of pregnancy, i.e. administration is usually only starting in the third or fourth week after fertilization.

Recently, a growing body of evidence has been accumulating that endogenous progesterone might play a significant role in establishing an adequate immune response during the early stages of successful pregnancy. The fetal-placental unit is a semi-allograft because of the paternal genetic contribution. The immunologic recognition of pregnancy and the subsequent activation of the maternal immune system is necessary for a successful pregnancy. In the presence of sufficient progesterone, for example activated lymphocytes among placental cells and decidual CD56+ cells synthesise the progesterone induced blocking factor (PIBF), a mediator which exerts substantial anti-abortive activities. PIBF alters the profile of cytokine secretion of activated lymphocytes. The production of non-inflammatory, non-cytotoxic interleukins (e.g., IL-3, IL-4, IL-10) is increased and the production of pro-inflammatory, cytotoxic cytokines (e.g., IFN-γ; TNF-α; IL-2) is reduced. PIBF inhibits the cytotoxicity of natural killer (NK) cells by blocking their degranulation and perform release. It also inhibits the IFN-γ, TNFα and IL-2 mediated transformation of NK cells to detrimental lymphokine activated killer (LAK) cells.

It is known that T helper cells (=Th cells) as essential components of the human cellular immunosystem are also playing a vital role in immunological disease states. Th cells can be differentiated in two different polarised forms which mobilise different types of effector responses, namely Th1 cells and Th2 cells. Th cells are able to produce and release (secrete) cytokines. The information processing within the immune system depends largely on the production and release of cytokines.

Concerning their impact on the immune response, cytokines can be divided into two categories. Th1 cytokines (e.g. Tumor Necrosis Factor (=TNF)α, Interferone-(=IFN)-γ, Interleukin (=IL)-2, IL-12, IL-18), as secreted by Th1 cells, induce several cell-mediated cytotoxic and inflammatory reactions. A Th1 immune response pattern is therefore dominated by a cytotoxic and inflammatory profile of cytokine release. Th2 cytokines (e.g., IL-4, IL-5, IL-6, IL-10, IL-13), as secreted by Th2 cells, are associated with B cell antibody production. Th2 cytokines downregulate Th1-type reactivity. A Th2 dominated immune response pattern is therefore dominated by an anti-inflammatory profile of cytokine release.

Certain immunological disease states are known to be associated with a specific pattern of pathological immune response, typically referred to as “Th1 immune response pattern”. Among such immunological disease states are e.g. autoimmune diseases like rheumatoid arthritis and multiple sclerosis or states of acute rejection response after organ and/or bone marrow transplantation. Activated lymphocytes in the placenta and peripheral blood mononuclear cells (=PBMC) in the peripheral blood are known to react in an immunomodulatory, i.a. anti-abortive way. The production of non-inflammatory, non-cytotoxic interleukins (e.g., IL-3, IL-4, IL-10; “Th2 pattern”) is increased and the production of pro-inflammatory, cytotoxic cytokines (e.g., IFN-γ; TNFα; IL-2, “Th1 pattern”) is reduced. For example, in successful pregnancy the normal profile is a Th2-type immunity while a shift towards Th1-dominance is thought to be associated with unexplained habitual abortion.

It is also known that endogenous progesterone inhibits in-vitro embryotoxic Th1 cytokine production to trophoblast in women with recurrent pregnancy loss (cf. B. C. Choi, K. Polgar, L. Xiao, J. A. Hill, Human Reproduction 15/1 (2000) 46-59). Progesterone does therefore seem to induce a shift from a Th1 immune response pattern to a Th2 immune response pattern in vitro.

However, progesterone is not a highly selective endogenous messenger. For example, progesterone, besides binding to the human progesterone receptor, is known to compete with glucocorticoids at glucocorticoid receptors. Besides, current metabolites of progesterone (e.g. 12 [symbol] -hydroxyprogesterone, androstenedione, testosterone, desoxycorticosterone, 11-desoxycortisol, corticosterone, cortisol, 18-hydroxycorticosterone and aldosterone) have a number of quite different activities. In particular, the corticosteroidal metabolites have a profound effect on the immune system. These corticosteroids are a group of steroids that affect carbohydrate metabolism (gluconeogenesis, liver glycogen deposition, elevation of blood sugar), inhibit corticotropin secretion, and even possess pronounced anti-inflammatory activity. They also play a role in fat and protein metabolism, maintenance of arterial blood pressure, alteration of the connective tissue response to injury, reduction in the number of circulating lymphocytes, and functioning of the central nervous system.

It has now surprisingly been found that dydrogesterone can influence Th1 dominated immune response patterns in a way that a stronger influence of a Th2 immune response pattern will prevail and that thereby dydrogesterone will exhibit a beneficial effect on immunological disease states which are characterised by a Th1 dominated immune response pattern.

Dydrogesterone is an orally active progestogen which is similar to endogenous progesterone in its molecular structure but, unlike progesterone, acts as a highly selective progesterone receptor agonist. Besides, metabolites of dydrogesterone are known to be either also selective agonists of human progesterone receptors or to be inactive. It can therefore be deduced that any physiological effects triggered by dydrogesterone will normally be mediated via the progesterone receptor. Dydrogesterone with its beneficial effects on immunological disease states which are characterised by a Th1 dominated immune response pattern can therefore serve as an example for the whole group of non-endogenous gestagen compounds which are acting as agonists for the human progesterone receptor. Due to the immunological role of said non-endogenous gestagen compounds in pregnancy that has been identified according to the present invention, it will be highly desirable to start administration of said non-endogenous gestagen compounds to female patients not only from the date of medical identification of pregnancy or even later, but already from the moment of ovulation on. Particularly advantageous will be the early administration of said non-endogenous gestagen compounds according to the invention to female patients that are exposed to an elevated risk of undergoing spontaneous or habitual miscarriage.

### SUMMARY OF THE INVENTION

The invention therefore relates to a method of treating or preventing spontaneous or habitual miscarriage which comprises administering to a female patient in need of such treatment an effective amount of at least one non-endogenous gestagen compound from the moment of ovulation on.

In a specific aspect of the invention, there is also provided a method for preselecting suitable female patients which are exposed to elevated risk of spontaneous or habitual abortion by identifying those female persons that respond to an appropriate stimulation with a pathological Th1 dominated immune response pattern. Said appropriate stimulation is preferably a mitogen-induced stimulation.

In yet another specific aspect of the invention, said female persons are identified by a process, comprising the steps of:
a) obtaining peripheral blood mononuclear cells (=PBMCs) from said female persons;
b) culturing said PBMCs with a protein extract from trophoblast;
c) assaying concentrations of Th1 and Th2 cytokines in the supernatant of the resulting PBMC culture, and
d) calculating the Th1/Th2 cytokine ratios for the respective female persons.

Suitable non-endogenous gestagen compounds which act as agonists of the human progesterone receptor are preferably selected from non-androgenising, non-endogenous gestagen compounds, which may typically be selected from the group consisting of desogestrel; dimethisterone; dydrogesterone; ethynodiol or ethynodiol diacetate; etonogestrel; gestodene; hydroxyprogesterone or hydroxyprogesterone caproate; keto-desogestrel; lynestrenol; medrogestone; medroxyprogesterone or medroxyprogesterone acetate; megestrol or megestrol acetate; nestorone; nomegestrol or nomegestrol acetate; norethynodrel; norgestimate; norgestrienone; promegestone; quingestanol or quingestanol acetate and trimegestone.

While dydrogesterone is a highly selective agonist for the human progesterone receptor, some of the other cited progestogens exhibit at least partial side effects like androgenic/antiandrogenic effects, estrogenic/antiestrogenic effects, glucocorticoid/antiglucocorticoid effects and/or mineralocorticoid/antimineralocorticoid effects. Further, in contrast to numerous other synthetic progestogens, dydrogesterone has no androgenic effects in the mother (e.g., hirsutism, voice changes, acne, etc.) and no masculinizing effect on the female fetus. Dydrogesterone is therefore preferred as non-endogenous gestagen compound.

The moment of ovulation can be determined by women according to known methods. Start of treatment from the moment of fertilization on will also have a favorable effect, but early starting from the moment of ovulation on is preferred.

The effects according to the invention of non-endogenous gestagen compounds, in particular of dydrogesterone, on Th1 dominated immune response patterns can be shown in a pharmacological test as set forth below.

Pharmacological Test Method

The effects of dydrogesterone on Th1 and Th2 cytokine profiles produced by mitogen-stimulated peripheral blood mononuclear cells (=PBMC) were established. The effects of dydrogesterone on Th1 and Th2 cytokine production were investigated in peripheral blood lymphocytes from women with unexplained habitual abortion. The peripheral mononuclear cells were stimulated with mitogen (phytohaemagglutinin) in vitro, in the presence and absence of dydrogesterone. The levels of cytokines secreted were measured after 96 hours in culture. Dydrogesterone strongly suppressed the production of the Th1cytokine IFN-gamma and increased the levels of IL-4 and IL-6.

Several studies have pointed to a deviation of cytokine profiles in women when during pregnancy. Pregnancy is associated with a shift towards Th2 bias, evinced by changes in peripheral T cells, by cytokine production by peripheral lymphocytes and by cytokine changes at the maternal-fetal interface. Unexplained recurrent spontaneous abortion (URSA) seems to be associated with a stronger bias towards Th1 cytokines. Women who have earlier been gone through URSA may therefore be regarded as a pre-selected group of patients that will typically respond to an immunological stimulation by showing a Th1 dominated pattern of immune response. The pharmacological test was therefore performed with a suitable URSA group which comprised of women admitted with spontaneous abortion for evacuation, (i) who have had at least two previous unexplained miscarriages, (ii) who were currently undergoing at least a 3rd abortion and (iii) who had been fully investigated.

The relationship between habitual abortion and cytokine production response in 54 women with normal pregnancy (and at least 3 previous normal pregnancies) and 23 women with a history of habitual abortion of unexplained aetiology (URSA group) has been investigated.

Mitogen-Induced Stimulation of Peripheral Blood Mononuclear Cells (PBMC):

Peripheral blood was obtained by venipuncture from the abortion group on the day of abortion. PBMC is separated by Ficoll-paque (Pharmacia Biotech, Sweden) density gradient centrifugation, suspended in RPMI medium (=“Roswell Park Memorial Institute”-medium; obtained from GIBCO/BRL, USA) containing 10% fetal calf serum, aliquoted into 96 well tissue culture plates at a density of 100.000 cells per well and then stimulated with the mitogen phytohemagglutinin (=PHA, Sigma Chemicals, USA) at a concentration of 5 mg/ml for a period of 96 hours, with autologous placental antigens or with trophoblast antigens. Progesterone, dydrogesterone (Duphaston®) and RU486 (=mifepristone) was added in different combinations as described below. Progesterone was tested at concentrations of 10⁻³ ml/L, 10⁻⁵ ml/L and 10⁻⁷ ml/L. RU486 was tested at equivalent concentrations. Culture supernatants were harvested at 96 hours for determination of the levels of cytokines secreted by the PBMC into the supernatants. Cytokine levels were determined in the supernatants by enzyme-linked immunosorbent assay (=ELISA).

### PROTOCOL OF EXPERIMENTAL SAMPLES

Each sample was tested in the following manner:
PBMC+mitogen
PBMC+mitogen+progesterone
PBMC+mitogen+progesterone+RU486
PBMC+mitogen+dydrogesterone
PBMC+mitogen+dydrogesterone+RU486
PBMC+mitogen+Carrier
PBMC+mitogen+Carrier+RU486
   Determination of cytokine levels by ELISA:

Th1 cytokines TNF-α and IFN-γ, and Th2 cytokines IL-4, IL-6 and IL-10 were evaluated in these samples by ELISA using kits that were obtained from Immunotech SA, France. These consisted of “sandwich ELISA”. Briefly, the first step leads to the capture of the relevant cytokine by monoclonal anti-cytokine antibodies bound to the wells of microtiter plates. In the second step, a second biotinylated monoclonal is added together with streptavidin-enzyme (peroxidase or alkaline phosphatase) conjugate. The biotinylated antibody binds to the solid phase antibody-antigen complex, and in turn, binds the conjugate. After incubation the wells are washed and the binding of the streptavidin-enzyme via biotin is followed by the addition of a chromogenic substrate. The intensity of the coloration produced is proportional to the concentration of the cytokine in the sample. Standard curves are plotted for each of the cytokines using reference recombinant cytokines and the results are read from these curves. The sensitivity of each of the assays is as follows: 5 μg/ml of TNF-α, 0.08 IU/ml of IFN-γ, 5 pg/ml of IL-4 and 5 pg/ml of IL-10. Results will be analysed by the t-test for independent samples.

There were significant differences between normal pregnant women and women with habitual abortion in terms of the levels of cytokines. Women with a normal pregnancy showed a higher Th2 bias, whilst women with a history of habitual abortion showed a bias towards Th1 reactivity. Results of the ex-vivo experiments according to the pharmacological test report given above showed that the administration of dydrogesterone (Duphaston®) in women with spontaneous abortion and habitual abortion reduced the activity of lymphocytes, resulted in a shift from a Th1 towards a Th2 bias (as evidenced by the reduction of IFN- [symbol] and a marked increase in Interleukins 4 and 6) and in the increased release of progesterone induced blocking factor (PIBF), a 34 kDA protein. PIBF is a molecular mediator within the subsequent immune response.

The results of the pharmacological test method given above can also be confirmed in an in vivo test model involving pregnant mice. In a murine stress-induced abortion model, a shift from Th1 to Th2-cytokine production was demonstrated. Pregnant mice were exposed to ultrasound on day 5 of gestation. This stress increased the abortion rate of these mice from around 12% to 44%. Treatment with dydrogesterone reduced the abortion in a dose-related manner to as low as 5%. The treatment resulted in an increased level of IL-4.

The non-endogenous gestagen compounds according to the invention may be administered in conventional pharmaceutical preparations. The doses to be used may vary individually and will naturally vary according to the type of condition to be treated and the identitiy of the active substance used. In general, however, suitable pharmaceutical dosage forms for administration to humans and larger mammals will contain 2.5 to 100 mg per individual dose per day, in particular when dydrogesterone is used as non-endogenous gestagen compound due to its good tolerability and safety. Usually an individual dose per day will contain 5 to 60 mg of a non-endogenous gestagen compound.

The compounds will preferably be administered orally, but can also be administered via other conventional routes usually used in hormone replacement therapy, e.g. intravaginally or transdermally. The compounds may be contained together with conventional pharmaceutical auxiliaries and/or carriers in solid or liquid pharmaceutical preparations. Examples of solid preparations are preparations which can be administered orally, such as tablets, coated tablets, capsules, powders or granules, or alternatively suppositories. These preparations may contain conventional pharmaceutical inorganic and/or organic carriers, such as talcum, lactose or starch, in addition to conventional pharmaceutical auxiliaries, for example lubricants or tablet disintegrating agents. Liquid preparations such as suspensions or emulsions of the active substances may contain the usual diluents such as water, oils and/or suspension agents such as polyethylene glycols and the like. Other auxiliaries may additionally be added, such as preservatives, taste correctives and the like.

The active substances may be mixed and formulated with the pharmaceutical auxiliaries and/or carriers in known manner. For the production of solid medicament forms, the active substances may for example be mixed with the auxiliaries and/or carriers in conventional manner and may be wet or dry granulated. The granules or powder can be poured directly into capsules or be pressed into tablet cores in conventional manner. These can be coated in known manner if desired.

Further examples of suitable pharmaceutical preparations are preparations for topical and/or transdermal delivery like gels, ointments or transdermal patches, devices and/or preparations for intravaginal administration, formulations for intranasal administration like sprays or formulations suitable for injection like depot injections or implants.

The foregoing description and examples have been set forth merely to illustrate the invention and are not intended to be limiting. Since modifications of the described embodiments incorporating the spirit and substance of the invention may occur to persons skilled in the art, the invention should be construed broadly to include all variations within the scope of the appended claims and equivalents thereof.

1. A method of inhibiting spontaneous or habitual miscarriage, said method comprising administering to a female patient in need of such treatment an effective amount of at least one non-endogenous gestagen compound from the moment of ovulation on.

2. A method according to claim 1, wherein the non-endogenous gestagen compound is non-androgenising.

3. A method according to claim 1, wherein the non-endogenous gestagen compound is selected from the group consisting of desogestrel; dimethisterone; dydrogesterone; ethynodiol; ethynodiol diacetate; etono-gestrel; gestodene; hydroxyprogesterone; hydroxyprogesterone caproate; keto-desogestrel; lynestrenol; medrogestone; medroxyprogesterone; medroxyprogesterone acetate; megestrol; megestrol acetate; nestorone; nomegestrol; nomegestrol acetate; norethynodrel; norgestimate; norgestrienone; promegestone; quingestanol; quingestanol acetate and trimegestone.

4. A method according to claim 1, wherein the non-endogenous gestagen compound is dydrogesterone.

5. A method according to claim 1, which comprises administering at least one non-endogenous gestagen compound in the time period between ovulation and medical identification of pregnancy.

6. A method according to claim 1, further comprising preselecting female patients who are exposed to elevated risk of spontaneous or habitual abortion by identifying those female persons that respond to stimulation with a pathological Th1 dominated immune response pattern.

7. A method according to claim 6, wherein the female persons are identified by a process comprising the steps of:
a) obtaining peripheral blood mononuclear cells (=PBMCs) from said female persons;
b) culturing said PBMCs with a protein extract from trophoblast;
c) assaying concentrations of Th1 and Th2 cytokines in the supernatant of the resulting PBMC culture, and
d) calculating Th1/Th2 cytokine ratios for the respective female persons.

8. A method according to claim 6, wherein said stimulation is a mitogen-induced stimulation.
